(19) United States
(12) Patent Application Publication
(10) Pub. No.: US 2026/0007321 A1
(43) Pub. Date: Jan. 8, 2026

(54) SYSTEM AND METHOD FOR SIMULTANEOUS ACQUISITION OF EIT AND ECG SIGNALS FROM THE SAME ELECTRODES

(71) Applicant: The Research Foundation for the State University of New York, Albany, NY (US)

(72) Inventors: Gary Saulnier, Albany, NY (US); Ahmed Abdelwahab, Morrisville, NC (US); Omid Rajabi Shishvan, Albany, NY (US); Nilton Barbosa da Rosa, Fort Collins, CO (US)

(21) Appl. No.: 19/259,217

(22) Filed: Jul. 3, 2025

Related U.S. Application Data
(60) Provisional application No. 63/667,278, filed on Jul. 3, 2024.

(51) Int. Cl. A61B 5/0536; A61B 5/00; A61B 5/28

(52) U.S. Cl. CPC A61B 5/0536; A61B 5/0044; A61B 5/28; A61B 5/7203; A61B 5/725; A61B 2576/023

(57) ABSTRACT

A system and method for simultaneous acquisition of electrocardiogram (ECG) signals and electrical impedance tomography (EIT) measurements on the same electrodes in a parallel-drive EIT system. A computer platform is in communication with a plurality of electrodes and applies currents to the electrodes and uses Howland noise reduction followed by an integrate-and-dump filter and adaptive filtering to extract the ECG signal without significant loss of amplitude.

### CROSS-REFERENCE TO RELATED APPLICATIONS

This application claims the benefit of U.S. Provisional Patent Application No. 63/667,278, filed on Jul. 3, 2024, the entirety of which is hereby incorporated herein by this reference.

### STATEMENT REGARDING FEDERALLY SPONSORED RESEARCH

This invention was made with government support under grant number 1 R01 EB026710-01A1, awarded by the National Institute of Biomedical Imaging and Bioengineering. The government has certain rights in the invention.

### BACKGROUND OF THE INVENTION

### 1. Field of the Invention

The present invention generally relates to medical devices. More particularly, the present invention relates to a system and method for the simultaneous acquisition of electrocardiogram (ECG) signals and electrical impedance tomography (EIT) measurements on the same electrodes in a parallel-drive EIT system.

### 2. Description of the Related Art

Electrical impedance tomography (EIT) is a non-ionizing imaging modality in which moderate frequency, low-amplitude currents are applied to electrodes on the surface of the body and the resulting voltages are measured on the electrodes. EIT signals are typically in the 10 kHz to 1 MHz frequency range. An inverse problem is solved numerically to compute the conductivity and permittivity distributions in the interior for each frame of data collected. With fast algorithms, images of air and blood volume changes can be computed and displayed in real time images of the chest. The high temporal resolution, portability, low cost, and absence of ionizing radiation make EIT particularly suitable for bedside pulmonary imaging.

Clinical studies have shown the utility of EIT to assess regional ventilation in patients with acute respiratory distress syndrome, as well as for lung perfusion assessment. Patients with chronic lung disease also require frequent lung monitoring and recent studies have demonstrated its potential for as-needed monitoring of patients with asthma, chronic obstructive pulmonary disease (COPD), cystic fibrosis, and spinal muscular atrophy.

The electrocardiogram (ECG) provides very important physiological signals in healthcare. ECG signals are low-frequency voltage potentials measured on the body's surface, typically in the frequency range of 0.05 Hz to 150 Hz. For bedside monitoring the ECG is typically measured through three electrodes placed on the patient's body. For diagnostic applications, a 12-lead ECG may be used. The electric dipole moment of the heart, produced by the flow of depolarization current through the heart tissue, is interrogated by measuring potential differences on the ECG electrodes as a function of time. Distinguished from EIT, no current is externally applied on the electrodes. The moving dipole moment can be reconstructed from the potential differences measured on the electrodes. Pathologies in the heart can be reflected in the ECG signal, resulting in its widespread use in cardiac diagnosis.

Previous studies have used cardiac-gated EIT to collect data, meaning that the EIT system collected data when it was triggered by the QRS complex of the ECG signal. Other EIT systems use commercial ECG recording devices for synchronous recording of EIT and ECG or have built-in support for ECG measurement in parallel to EIT signals. These ECG recording systems require either an independent system, or extra circuit modules for functioning and must use separate electrodes in addition to the ones used for EIT for data collection. In one study, an interleaved EIT system is introduced where the voltage sensors that collect the EIT voltages can simultaneously measure ECG signals. ECG signals are isolated from the EIT signals by filtering the measured voltage using a first order high-pass filter at 0.05 Hz followed by second order low-pass filter at 150 Hz.

Accordingly, it would be advantageous to provide a system or method that can use the same electrodes to simultaneously measure ECG and EIT data from a patient. It is thus to such a system and method that the present invention is primarily directed.

### BRIEF SUMMARY OF THE INVENTION

Briefly described, the present invention provides a system and method system and method for simultaneous acquisition of ECG signals and EIT measurements on the same electrodes in a parallel-drive EIT system. A computer platform is in communication with a plurality of electrodes and applies currents to the electrodes using a Howland current-source with noise reduction followed by an integrate-and-dump filter and adaptive filtering to extract the ECG signal without significant loss of amplitude.

The present integrated EIT/ECG system eliminates the need for a separate system to collect ECG signals when performing EIT measurements, including the need for additional electrodes. Having time-aligned ECG signals is helpful in interpreting EIT images, particularly when imaging pulsatile perfusion images in the thorax since the exact timing of each image in relation to the cardiac cycle is known. Additionally, collecting ECG signals from some or all of the EIT electrodes enables reconstruction of the moving dipole moment at the same time as the EIT images.

In an embodiment, the present system and method simultaneously acquire and display in real-time the ECG signals and EIT images in an ACT 5 electrical impedance imaging system. This system is a parallel-drive EIT system that applies alternating current at frequencies in the range from 5 kHz to 500 kHz and measures the resulting voltages on all electrodes simultaneously at a frame rate of ƒ_{FR}≈27 frames/sec on 32 electrodes. As ACT 5 is a parallel-drive EIT system where currents are applied to and voltages are measured on all electrodes simultaneously, ECG waveforms are recovered from electrodes that are connected to a current source and are actively delivering EIT current. The analog circuits can be used within the ACT5 system including those to the conventional Howland current source, for applying EIT signals while preserving ECG signals. The recovered ECG waveforms from 16 to 32 electrodes can be used to reconstruct the heart's current source vectors during a cardiac cycle, yielding an approximate solution to the inverse problem of electrocardiography.

In one embodiment, the invention provides a system for collecting electrocardiogram (ECG) signals and electrical impedance tomography (EIT) measurements from the same electrodes on a patient, with a computer platform and a plurality of electrodes in communicative connection to the computer platform, the plurality of electrodes configured to be placed on a patient. The computer platform is further configured to apply a set of currents to the plurality of electrodes to acquire both ECG and EIT signals and produce one or more diagnostic images of the patient.

In another embodiment, the invention provides a method for collecting electrocardiogram (ECG) signals and electrical impedance tomography (EIT) measurements from the same electrodes on a patient, with the steps of applying, from a computer platform, a set of currents to a plurality of electrodes, the plurality of electrodes in communicative connection to the computer platform, the plurality of electrodes placed on a patient, and then applying, from the computer platform, a set of currents to the plurality of electrodes to acquire both ECG and EIT signals therefrom. The method continues by producing, at the computer platform, one or more diagnostic images of the patient.

The present invention accordingly provides an advantage in allowing EIT and ECG data to be obtained from a patient from the same set of electrodes. The present invention is industrially applicable as it is a medical device that can be manufactured with this novel capability. Other features, advantages, and objects of the present invention will be apparent to one of skill in the art after review of the present application.

### BRIEF DESCRIPTION OF THE DRAWINGS

FIG. 1A is a circuit diagram of a simple Howland current source structure.

FIG. 1B is a circuit diagram of a modified Howland current source with a capacitor on the positive feedback path to minimize the Howland noise on the ECG signal.

FIG. 2 is a circuit diagram of a modified Howland current source with a series capacitor to the load.

FIG. 3 is a graph illustrating the magnitude of the output impedance of the basic Howland, the Howland with C_{c} and C_{f} but without R_{L}, and the Howland source with C_{c}, C_{f}, and R_{L}.

FIG. 4 is a graph of spectral density of the output noise for the basic Howland, the modified Howland with C_{c} only, and the modified Howland with both C_{c} and C_{f}.

FIG. 5 is a block diagram of one embodiment of the DC removal system.

FIG. 6 is a circuit diagram of one embodiment to an adaptive least mean square filter.

FIG. 7 is a block diagram of one embodiment of the simultaneous EIT/ECG system.

FIG. 8A is a picture of one embodiment of a 2×16 electrode arrangement on the chest of a patient.

FIG. 8B is a picture of one embodiment of a 2×16 electrode arrangement on the back of a patient.

FIG. 9A is a series of graphs illustrating a comparison between raw and filtered ECG signals without applying EIT current patterns.

FIG. 9B is a series of graphs illustrating a comparison between raw and filtered ECG signals while applying EIT current patterns.

FIG. 10 is a series of ECG waveforms obtained from the upper ring of electrodes.

FIG. 11 illustrates pulsatile perfusion images with an ECG trace at the beginning of the P wave, the start of atrial depolarization.

FIG. 12 illustrates pulsatile perfusion images with an ECG trace at the end of QRS complex, at the end of the diastolic phase before the blood ejection from heart to lungs.

FIG. 13 illustrates pulsatile perfusion images with an ECG trace at the beginning of the T wave, the start of ventricular repolarization.

FIG. 14 illustrates pulsatile perfusion images with an ECG trace at the end of the T wave, the end of ventricular repolarization.

FIG. 15A is a graph of the comparison of the total cardiac vector paths for a single subject when simultaneously collecting EIT and ECG data.

FIG. 15B is a graph of the comparison of the total cardiac vector paths for a single subject when collecting only ECG data.

### DETAILED DESCRIPTION OF THE INVENTION

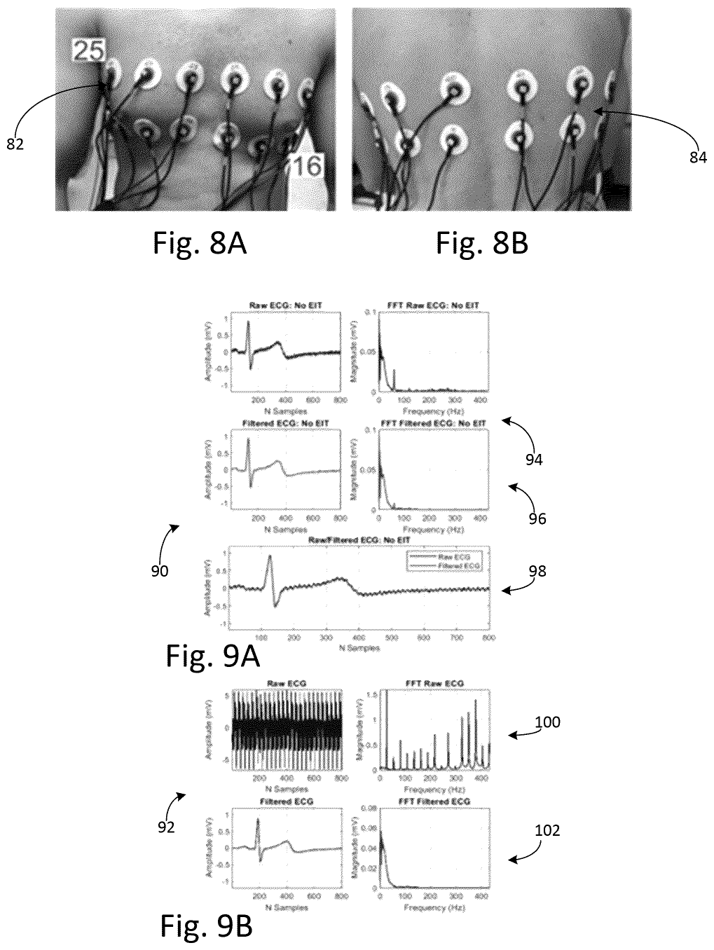

With reference to the figures in which like numerals represent like elements throughout the several views, the present invention acquires and displays, in real-time or a substantially rapid manner, the ECG signals and EIT images in an ACT5 electrical impedance imaging system. The Adaptive Current Tomograph 5 (ACT5) Electrical Impedance Tomography (EIT) system is a 32 electrode (FIGS. 8A,8B) applied-current multiple-source EIT system that can display real-time images of conductivity and susceptivity at 27 frames per second. The adaptive current sources in ACT5 can apply fully programmable current patterns with frequencies varying from 5 kHz to 500 kHz. The system also displays real-time ECG readings during the EIT imaging process. The ACT5 system is more thoroughly described in “ACT5 Electrical Impedance Tomography System,” IEEE Trans Biomed Eng. 2023 Dec. 22; 71(1):227-236, the entirety of which are hereby incorporated herein by this reference.

In an embodiment, the system uses an ACT5 system as the parallel-drive EIT system that applies alternating current at frequencies in the range from 5 kHz to 500 kHz and measures the resulting voltages on all electrodes simultaneously at a frame rate of ƒ_{FR}≈27 frames/sec on 32 electrodes. As ACT 5 is a parallel-drive EIT system where currents are applied to and voltages are measured on all electrodes simultaneously, ECG waveforms are recovered from electrodes that are connected to a current source and are actively delivering EIT current.

During the imaging process of the ACT 5 system, electrodes (82,84, FIGS. 8A,8b) are attached to the chest and are continuously delivering EIT currents and measuring the voltages on the body surface. The aim is to recover ECG waveforms from these measured voltages simultaneously with the EIT imaging process. Recovering the ECG signal from the combined EIT/ECG signal is aided by the relatively large frequency difference between the ECG and EIT signals, but the presence of the EIT current source introduces two problems: (i) the introduction of noise into the ECG signal and (ii) the attenuation of the ECG signal due to the loading introduced by the current source output impedance. The current sources in ACT 5 compensate for current loss through shunting impedances, including those introduced by the cables between the sources and electrodes, using knowledge of the shunting impedances at the EIT excitation frequency and the measured voltage. A digital control loop automatically adjusts the applied EIT current to offset the current lost in the shunting impedance. The output current is provided by a basic Howland current pump, though it is not necessary for the Howland source to maintain a high intrinsic output impedance because its output impedance, like any other shunt impedance, will be compensated for by the digital control loop.

When delivering current to a load, the Howland source will also inject a noise current due to the inherent noise of the op amp, resistive elements in the circuit, and the conversion of noise in the input signal into a current. This current noise will flow in the total load that consists of the load impedance in parallel with the output impedance of the source to produce a noise voltage. The 1/ƒ noise from the op amp and driving signal will dominate this noise voltage at low frequencies and, consequently, be added to the ECG signal and reduce its signal-to-noise ratio (SNR).

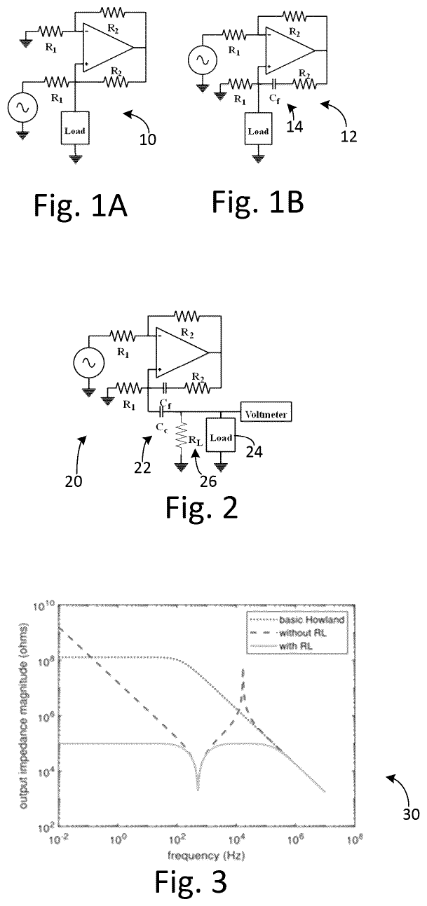

An approach to reduce this noise contribution is to lower the output impedance and gain of the Howland source at low frequencies by inserting a series capacitance into its positive feedback path. FIG. 1A is a circuit diagram 10 of a simple Howland current source structure and shows the basic Howland source.

FIG. 1B is a circuit diagram 12 of a modified Howland current source with a capacitor on the positive feedback path to minimize the Howland noise on the ECG signal and a modified version that includes this capacitor 14 (C_{f}) in the positive feedback path. Note also that the input has been moved to the upper R₁ which makes the source inverting. Assuming an ideal op amp, the resulting gain A(jƒ) and output impedance Z₀(jƒ) of the modified Howland source are

$$A ( jf ) - \frac{1}{{R}_{1}} [ \frac{j 2 π {fC}_{f} {R}_{2}}{1 - j 2 π {fC}_{f} {R}_{2}} ]$$

$$and$$

$${Z}_{0} ( jf ) = {R}_{1} [ 1 + j 2 π {fC}_{f} {R}_{2} ] ,$$

The basic Howland with perfectly balanced resistors has a gain of 1/R₁ and an infinite output impedance. For the modified circuit, the above shows that the gain has a zero at DC and a pole at ½πC_{f}R₂, resulting in zero gain at DC that transitions to a flat gain at high frequencies. Having the gain drop off at low frequencies will reduce the low frequency current noise at the Howland output that originates from the circuit driving the Howland. The output impedance given by the lower equation has a zero at ½πC_{f}R₂, indicating that the output impedance is R₁ at DC and goes up as frequency increases.

Reducing the Howland output impedance and gain at low frequencies lowers the noise in the ECG frequency range. An unfortunate side-effect, however, is the potential reduction of the ECG signal amplitude due to the loading effect of the small Howland output impedance. One way to retain high impedance at low frequencies is to insert a coupling capacitor C_{c} between the Howland source and the load, as shown in FIG. 2.

FIG. 2 is a circuit diagram 20 of a modified Howland current source with a series capacitor 22 (C_{c}) to the load 24. The value for C_{c} must be large enough to pass the EIT frequencies without excessive voltage drop and a corresponding reduction in voltage compliance while also providing a sufficiently high impedance at the ECG frequencies. Also, a coupling capacitor alone will present an impedance that continues to increase as frequency decreases, resulting in the ECG signal seeing an impedance that varies across its frequency band. To flatten the impedance over the ECG band, a resistor 26 R_{L} is placed in parallel with the source as shown in FIG. 2. This parallel resistor 26 limits the maximum impedance seen looking back at the source for both the ECG and EIT signals. The adaptive loop in the ACT5 current source system compensates for shunt impedance at the EIT excitation frequency, meaning that it will not result in current loss for the EIT signal.

FIG. 3 is a graph 30 illustrating the magnitude of the output impedance of the basic Howland, the Howland with C_{c} and C_{f} but without R_{L}, and the Howland source with C_{c}, C_{f}, and R_{L}. The basic Howland source has very high output impedance at low frequencies which drops off at 20 dB/decade above 100 Hz due to the output capacitance. Introducing C_{c} and C_{f} results in the output impedance continuing to increase as frequency decreases due to the series impedance of C_{c}. Adding R_{L}=100 kΩ, which is in parallel with the output impedance presented by the modified Howland in series with C_{c} limits the output impedance to 100 kΩ, producing the desired flat impedance through the ECG frequency band, with it rolling off by 3 dB at 144 Hz.

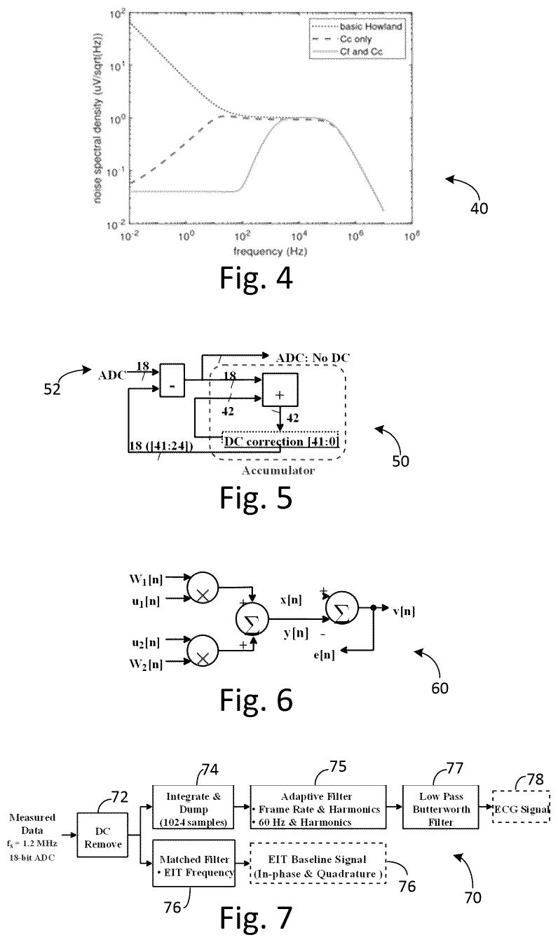

FIG. 4 is a graph 40 of spectral density of the output noise for the basic Howland, the modified Howland with C_{c} only, and the modified Howland with both C_{c} and C_{f}. FIG. 4 shows the spectral density of the noise at R_{L} from the simulation for the basic Howland, the Howland with the addition of C_{c} only, and the Howland with both C_{c} and C_{f} in units of ρV/√Hz. The result for the basic Howland shows the strong 1/ƒ noise component that will introduce significant noise into the ECG signal. Adding C_{c}=10 nF greatly reduces the low frequency noise, but the spectral density of the noise voltage is still large near the upper end of the ECG band at 150 Hz. Note that the addition of C_{c} alone required a slight imbalancing of the Howland to reduce its output impedance at DC to accommodate the small output current due to the bias currents and offset voltage of the op amp. The imbalance was introduced by making the R₁ at the lower left equal to 1996Ω rather than 2 kΩ. This imbalancing is not needed once C_{f} is added because it makes the Howland gain zero at DC. The combination of C_{c} and C_{f} results in near complete suppression of the noise contribution from the Howland in the ECG band. The residual noise spectral density at 1 Hz is 40.72 nV/√Hz, which is just slightly above the thermal noise of the 100 kΩ resistor at room temperature, √4 kTR=40.57 nV/√Hz at 25° C., indicating that the Howland source is not contributing significant noise to the output. The RMS noise in the frequency range from 0.01 Hz to 150 Hz is 20.819ρV for the basic Howland, 12.318ρV with C_{c} alone, and 622.31 nV with both C_{c} and C_{f}, representing a 30.5 dB reduction in noise from the basic Howland to the final design.

The modifications to the Howland source produce the desired low noise and flat impedance up to approximately 150 Hz. However, the impedance in this range is well below the 10 MΩ prescribed by IEC60601-2-47 for diagnostic ECG instruments which could result in some attenuation of the ECG signals. This compromise is necessary to enable ACT 5 to obtain good quality ECG signals while simultaneously applying currents for EIT measurements. ACT 5 is a parallel-drive EIT system that applies currents to all electrodes simultaneously. For each frame of image, a set of orthogonal current patterns is applied, where each pattern defines the current for each electrode, to produce the data for one image. The 32 electrode ACT 5 system applies 32 orthogonal current patterns, measuring the voltages on each electrode for each current pattern, to produce one image frame. For each electrode, the applied current waveform consists of a series of sinusoidal bursts, with each burst having an amplitude and phase corresponding to the current to be applied to that electrode for one pattern.

The analog-to-digital converters (ADCs) in ACT 5 sample the electrode voltages at 1.2 MSample/s. The first step of processing the voltage is removing its DC component. Since the ADC is continuously measuring the voltages, the DC component can be removed by passing the measured voltages through a digital high-pass filter shown in FIG. 5. FIG. 5 is a block diagram 50 of one embodiment of the DC removal system. The infinite impulse response (IIR) filter can be described by the following difference equation:

$$y [ n ] = x [ n ] - \frac{{\sum }_{k = 1}^{n - 1} y [ k ]}{{2}^{24}} .$$

where n is the sample number, x[n] is the ADC sample 52, and y[n] is the output of the filter. This filter has a first order high pass response with a −3 dB frequency of 0.018 Hz, which does not interfere with ECG signals and only affects DC and other extremely low frequency signals.

For EIT voltages, ACT5 uses quadrature matched filters operating over 1024 samples to recover the in-phase and quadrature (real and imaginary) voltages. If y[n] is the sampled voltage waveform and Tis the sampling interval, the measured in-phase (V_{I}) and quadrature (V_{Q}) voltages are

$${V}_{I} = {\sum }_{n = 1}^{1024} y [ {n}^{\text{?}} ] cos ( ω nT )$$

$${V}_{Q} = {\sum }_{n = 1}^{1024} y [ {n}^{\text{?}} ] sin ( ω nkT ) .$$

$$\text{?} \text{indicates text missing or illegible when filed}$$

The excitation frequency, ω, is chosen such that 1024 samples contains an integral number of cycles of the sinusoid, making V_{I} and V_{Q} insensitive to any DC offset in y[n].

Although the relatively large frequency difference between the EIT and ECG signals should make the separation easy, several factors make separation more difficult. The high sampling rate of 1.2 MHz used in ACT5 compared to the desired bandwidth of 150 Hz for the ECG signal would require the use of high precision coefficients for a digital low pass filter in a fixed-point format. Down-sampling could mitigate this issue, but ACT 5 can use several discrete excitation frequencies in the range from 5 kHz to 500 kHz, making aliasing of the EIT signal into the ECG band a potential problem at some frequencies. Another critical factor is the large magnitude difference between the EIT and ECG signals. The ACT5 system is designed to allow a maximum swing of 1 Vpp for the measured EIT voltage, which is three orders of magnitude higher than the expected ECG signal that is in the mV range. The EIT signal must be heavily attenuated to recover an ECG with a good signal-to-interference ratio. As noted earlier, the EIT excitation frequencies are selected such that an integral number of cycles will occur over 1024 samples at the 1.2 MSample/sec sampling frequency. As a result, the sum of 1024 samples of the EIT signal at any of these frequencies equals zero. An integrate-and-dump filter operating over 1024 samples in parallel with matched filters will remove the EIT signal while preserving the ECG signal. The integrate-and-dump filter has a sin(x)/x frequency response with nulls at each possible EIT frequency. The recovered ECG signal, V_{ECG}, is

$${V}_{ECG} = {\sum }_{n = 1}^{1024} y [ k ] ,$$

where y[k] now denotes the combined EIT/ECG signal. With this approach, one sample of the ECG signal is produced when each burst of the EIT signal is applied, meaning that the ECG sampling rate is the same as the EIT frame rate times the number of current patterns applied for each frame. The sampling rate in the default configuration is approximately 864 samples/sec. While the integrate-and-dump filter completely removes frequency components at the EIT excitation frequency and its harmonics, it still passes interfering frequency components related to the frame rate (FIG. 7).

Like most EIT systems, ACT5 uses DC blocking capacitors between the electronics and the electrodes to ensure that a DC signal cannot be applied to the subject. These capacitors may be left with some residual charge at the end of each sinusoidal burst of the EIT signal, creating frequency components at the pattern repetition rate, i.e. the EIT frame rate, and its harmonics. Some of these frequencies are below 150 Hz and, therefore, fall inside the ECG bandwidth. The integrate-and-dump filters also fail in blocking any power line interference noise on the measurements. Since the frame rate and the power line frequency are known, digital notch filters could be used to suppress that frequency and its harmonics. However, the standard finite and infinite impulse response filters cannot remove the harmonics without attenuating frequencies from the ECG signal because of limited quality factors, making them unfeasible. A better approach is to use an adaptive filter to subtract components at the frame rate and its harmonics as well as the USA power line frequency of 60 Hz and its harmonics, up to 150 Hz. This approach removes only the interfering frequency components, effectively implementing a set of zero bandwidth notch filters. The implementation of the adaptive filter uses the least mean square (LMS) algorithm to minimize the residual signal at the interfering frequencies.

FIG. 6 is a block diagram 60 of one embodiment to an adaptive least mean square filter. FIG. 6 shows the block diagram of an LMS adaptive filter that can be used to remove a tone of known frequency. The reference signals u₁[n] and u₂[n] are multiplied by the adaptive tap weights W₁[n] and W₂ to produce y[n]y[n]:

$$y [ n ] = {W}_{1} [ n ] {u}_{1} [ n ] + {W}_{2} [ n ] {u}_{2} [ n ] ,$$

$$where$$

$${u}_{1} [ n ] = {A}_{c} cos ( 2 π {f}_{k} {nT}_{s} )$$

$${u}_{2} [ n ] = {A}_{c} sin ( 2 π {f}_{k} {nT}_{s} )$$

and ƒₖ is the frequency of the sinusoidal interferer, Tₛ is the ECG sampling interval, and n is the time increment. y[n] is subtracted from the primary signal x[n] which, in this case, is the ECG signal with the interfering tone. With the reference signals given by the above equation, the weights can synthesize a sinusoid of arbitrary amplitude and phase at frequency ƒₖ. The adaptive filter tap weights (W[n+1]) are adapted using

$${W}_{1} [ n + 1 ] = {W}_{1} [ n ] + μ e [ n ] {u}_{1} [ n ]$$

$${W}_{2} [ n + 1 ] = {W}_{2} [ n ] + μ e [ n ] {u}_{2} [ n ] ,$$

where μ is the step-size parameter and e[n]=x[n]−y[n] is the error signal. In adjusting the weights to minimize the error signal e[n], the adaptive filter suppresses the tone at ƒₖ. The filter output v[n] equals the error signal.

Since the adaptive LMS filter approach shown in FIG. 6 removes a single interfering frequency, to suppress all harmonic interfering frequencies (ƒₖ), adaptive LMS filters were implemented for each ƒₖ and then cascaded where the primary signal for each subsequent filter is the filter output v[n] from the previous stage and the reference signals u₁[n] and u₂[n] are the sine and cosine of the next frequency to be suppressed.

FIG. 7 is a block diagram 70 of one embodiment of the simultaneous EIT/ECG system. FIG. 7 shows all stages for ECG signal extraction from EIT in which the approach to recovering the ECG signal takes advantage of the EIT signal design. After DC is removed, block 72, the integrate-and-dump filter 74 integrates over 1024 samples to remove the EIT frequencies. After the integrate-and-dump filter 74 the adaptive filter, block 75, removes the frame rate and line frequencies and their harmonics below 150 Hz. After the adaptive filter 75, a Butterworth lowpass filter, block 77, removes frequencies above 150 Hz. The output of the lowpass filter is the recovered ECG signal, 78. Using a parallel path, the output of block 72 is processed by matched filters, block 76, to recover the baseband EIT signals, 76.

In one embodiment, the adaptive filter 75 suppresses the frame rate frequency ƒ_{FR} and the 60 Hz line frequency and their harmonics below 150 Hz. The LPF Butterworth filter 77 suppresses the remaining harmonic interfering frequencies above 150 Hz as well as high-frequency noise. The LPF Butterworth filter 77 implemented using the filtfilt function from MATLAB, with a cutoff frequency of 150 Hz, was used as a final stage of filtering for the received ECG signal 78. Moreover, the lower cutoff frequency is set to 0.159 Hz by the AC coupling prior to the ADC in ACT5. Additionally, Table 1 provides a detailed list of interference frequencies on the ECG signal and how they are removed via digital signal processing.

TABLE 1

| Frequency (Hz) | Description | How removed |
|---|---|---|
| 0-0.159 | DC and low frequency | AC coupling |
| | effects | |
| f_{FR} & harmonics | Frame rate-related noise | Adaptive filter |
| 60 & harmonics | Power line noise | Adaptive filter |
| EIT excitation | EIT sinusoid noise | Integrate & dump |

Thus, in one embodiment, the invention provides a system, such as that shown in FIG. 7, for collecting electrocardiogram (ECG) signals and electrical impedance tomography (EIT) measurements from the same electrodes (electrodes 82,84; FIGS. 8A,8B) on a patient, with a computer platform embodying the elements of the block diagram 70 and performing the functions stated above therefore. The invention can, but not necessarily, also include the plurality of electrodes (82,84) that are in communicative connection to the computer platform, with the plurality of electrodes configured to be placed on a patient as shown in FIGS. 8A,8B. The computer platform is configured, in hardware or in software, to apply a set of currents to the plurality of electrodes to acquire both ECG and EIT signals and produce one or more diagnostic images of the patient, as is further shown and described herein.

Accordingly, the present invention can be a stand-alone device containing a computer platform as described herein, such as an ACT5 system. Alternately, the computer platform described herein can be distributed across several physical or virtual computer components that can perform some or all of the functions described herein.

FIG. 8A is a picture of one embodiment of a 2×16 electrode arrangement 82 on the chest of a patient. FIG. 8B is a picture of one embodiment of a 2×16 electrode arrangement 84 on the back of a patient. EIT and ECG data were collected in accordance with the amended Declaration of Helsinki-Ethical Principles for Medical Research Involving Human Subjects under the approval of the Colorado State University Institutional Review Board (approval number 2943) with written informed consent. The data were collected on a 35-year-old healthy male subject having a body mass index (BMI) of 28.4 and a chest circumference of 100.3 cm. The subject was in the supine position with 2 rows of 16 electrodes attached around his thorax (FIG. 8A) with the top row placed above the nipple line and the bottom row below the nipple line, and with the approximation for lead II electrocardiogram taken to be the voltage difference between electrodes 16 and 25 (FIG. 8A). As shown here, 32 electrodes can be used, but greater or lesser amounts of electrodes can also be used as would be apparent to one of skill in the art. For purposes of the present embodiment, 16 to 32 electrodes is satisfactory.

First, ECG data was collected without applying EIT to evaluate the filter stage. The adaptive filters were implemented to remove each interfering frequency with μ=0.001, A_{c}=1, ƒₖ=ƒ_{FR}k, with k={1, . . . ,11}. In addition, due to USA power line interference, we included the first five harmonics of 60 Hz in the adaptive filter stage, with ƒₖ=60 k and k={1, . . . ,5}. Thus, nineteen harmonic frequencies are attenuated using the adaptive filter. Moreover, for the low-pass filter stage, we implemented the Butterworth filter of order 40 with the cutoff frequency of 150 Hz using the filtfilt function in the MATLAB toolbox.

FIG. 9A is a series of graphs 90 illustrating a comparison between Raw and Filtered ECG signals without applying EIT current patterns. FIG. 9B is a series of graphs 92 illustrating a comparison between raw and filtered ECG signals while applying EIT current patterns. FIG. 9A shows the comparison between the raw and filtered ECG signals for lead II without applying EIT current patterns. The top image 94 shows the raw ECG signal and its harmonic components. In this case, we can see the powerline 60 Hz frequency interference present in the FFT plot. The middle image 96 illustrates the filtered ECG with an attenuated 60 Hz interference in the FFT plot while the bottom image 98 shows both raw and filtered ECG displayed on the same plot which shows that the filter stage is targeting the interfering harmonic frequencies with minimal signal attenuation and without delaying the ECG signal.

For the second experiment, EIT currents were applied and both EIT and ECG data were collected. For EIT signal, a tensor product of trigonometric patterns with a peak current of 0.35 mA and an applied current frequency of 93.750 kHz were used during breath-holding. FIG. 9B shows the comparison between the raw and filtered ECG signals when applying EIT current patterns. The top image 100 shows the raw ECG signal and its harmonic components, including the interfering frequencies from EIT and powerline. The bottom image 102 shows the ECG signal and its harmonics after the adaptive and low-pass filter stages. This demonstrates that the filter stage is suppressing the interfering frequencies effectively, resulting in a clean ECG signal.

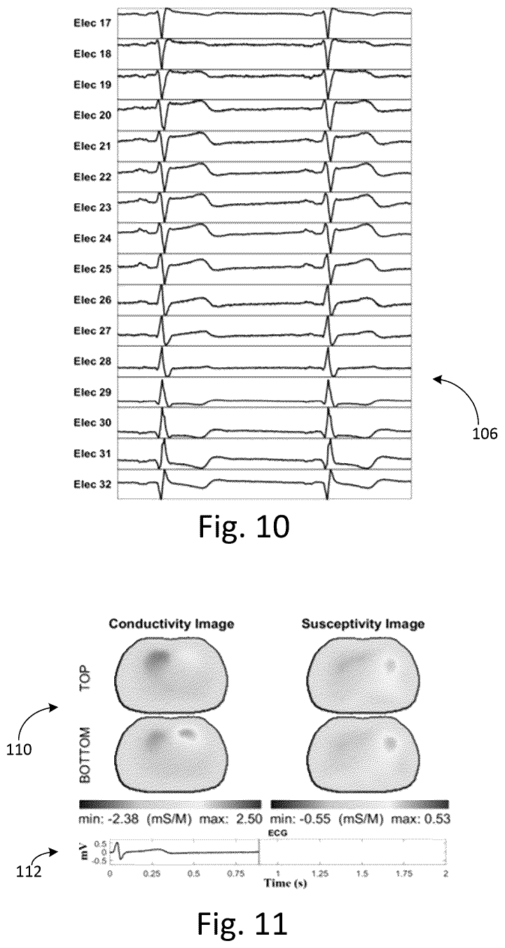

FIG. 10 is a series of waveforms 106 obtained from the upper ring of electrodes. FIG. 10 shows the normalized ECG signals measured on each of the electrodes of the upper ring, electrodes 17 through 32. The waveforms were produced by processing the voltage from each electrode according to the block diagram 70 of FIG. 7, finding the average of the waveforms from all 32 electrodes, and subtracting this average waveform from those for electrodes 17 through 32. Although ECG waveforms for all 32 electrodes are available, only those from the top ring are displayed here.

3-D reconstructions from the data collected using the ACT5 system were computed using the ToDLeR algorithm. ToDLeR is a linearized reconstruction algorithm derived for a cylindrical model of the chest that assumes the conductivity is a small perturbation from a reference state. The computations were performed on a cylindrical domain with the radius and height derived from the subject's chest circumference, and then the reconstructed conductivity and susceptivity on each circular cross-section were conformally mapped to a chest shape. The reconstructions of pulsatile perfusion images were computed from short time intervals during breath-holding.

FIGS. 11-14 show the EIT reconstructions of pulsatile perfusion from simultaneously collecting both EIT and ECG data, with the moment in the cardiac cycle that represents each reconstruction indicated by the vertical red line in the ECG plot. The EIT reconstructions are difference images after drift removal, with the average of all frames in a 250 frame sequence chosen as the reference. The images are single frames without post-processing noise filtering or averaging and are displayed in DICOM orientation. The reconstructions show conductivity and susceptivity images for slices at the levels of the top and bottom electrode rings (FIGS. 8A, 8B).

FIG. 11 shows pulsatile perfusion images 110 with an ECG trace 112 at the beginning of the P wave, the start of atrial depolarization. At the beginning of the P wave 112 (FIG. 11), the ventricles are filling and the heart has a higher conductivity and susceptivity than the lungs. The heart appears primarily in the lower slice.

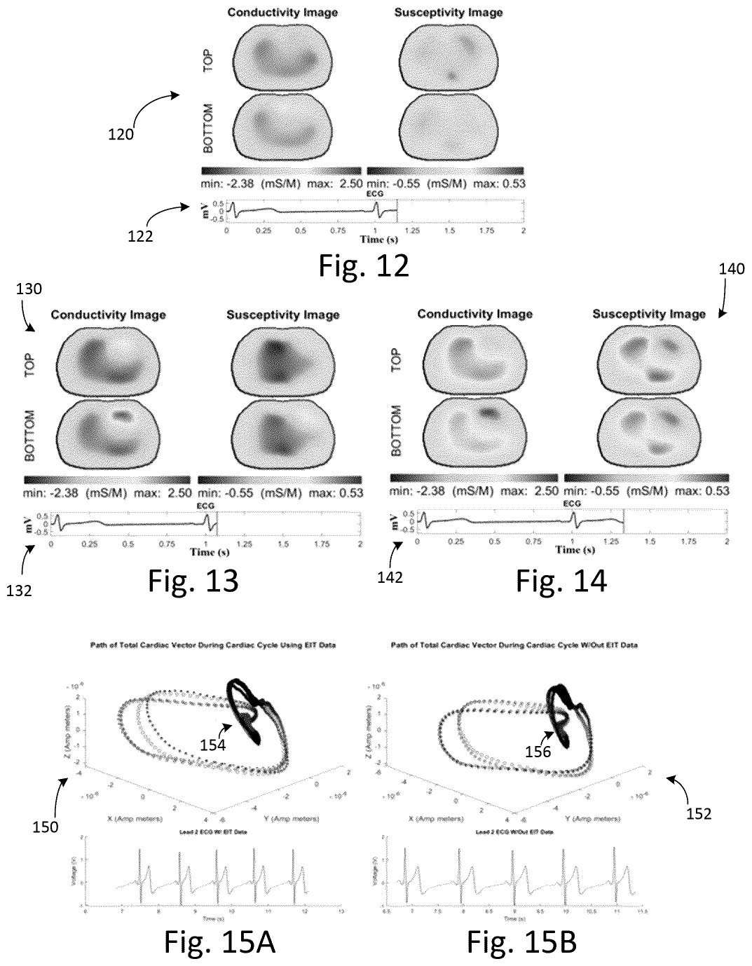

FIG. 12 illustrates pulsatile perfusion images 120 with an ECG trace 122 at the end of QRS complex, at the end of the diastolic phase before the blood ejection from heart to lungs. End-diastolic volume is reached at the end of the QRS complex (FIG. 12) which is the moment that the heart pixels show the maximum conductivity and susceptivity values while the pixels in the regions of the lungs show the lowest conductivity and susceptivity values.

FIG. 13 illustrates pulsatile perfusion images 130 with an ECG trace 132 at the beginning of the T wave, the start of ventricular repolarization. Later, at the beginning of the T wave, the conductivity and susceptivity in the heart decreases while they increase in the lungs due to the blood ejection from the right ventricle into the pulmonary artery. FIG. 14 illustrates pulsatile perfusion images 140 with an ECG trace 142 at the end of the T wave, the end of ventricular repolarization. The end-systolic volume is reached at the end of the T wave, which is the moment that the pixels in the lung regions have the highest conductivity and susceptivity values while the heart pixels show the lowest conductivity and susceptivity values before the ventricular filling starts again. The simultaneous measurement of EIT and ECG data by ACT5 allows us to solve the inverse problem of electrocardiography. The inverse problem of electrocardiography is to reconstruct the heart's current density source, \vec{J}H(\vec{x}, t) inside the heart, H, given the ECG voltages, V(\vec{x},t), that it induces on the body's surface, S. To reconstruct J^{-H} we use the conductivity, σ(\vec{x},t), throughout the chest, Ω, reconstructed from the simultaneously measured EIT data. We assume the voltage measurements on the body's surface at time t, that arise from the electrical activity of the heart satisfy the following “Forward Model of electrocardiography.” Let Ω denote the body, S the boundary of Ω, and H⊂Ω denotes the heart.

The ECG voltages V(\vec{x},t)=u(\vec{x},t)|_{\vec{x}∈}s, where u(\vec{x},t) satisfies the Boundary Value Problem:

$$∇ \cdot [ σ ( {x}^{\text{?}} , t ) ∇ u ( {x}^{\text{?}} , t ) = ∇ \cdot {J}^{H} ( {x}^{\text{?}} , t ) {x}^{\text{?}} ∈ Ω$$

$$σ ( {x}^{\text{?}} , t ) \frac{\partial u}{\partial \hat{n}} ( {x}^{\text{?}} , t ) = 0 {x}^{\text{?}} ∈ S$$

$$\text{?} \text{indicates text missing or illegible when filed}$$

Here, the heart's current sources are modeled by a single dipole located at a point \vec{Q}₀, inside the heart, H, by \vec{J}H(\vec{x}, t)=\vec{M}(t)δ(\vec{x}−\vec{Q}₀). This approximation implies that the dipole moment M^{→}(t) is equal to the total cardiac vector which is defined to be the total current density over the heart at a given time t:

$${M}^{\text{?}} ( t ) := {\int }_{H} {J}^{H} ( {x}^{\text{?}} , t ) d {x}^{\text{?}} .$$

$$\text{?} \text{indicates text missing or illegible when filed}$$

Given the conductivity and the position of the dipole source, the inverse ECG problem becomes the linear problem of finding \vec{M}(t) from the ECG voltages, V(t), on the body's surface at each time t. This is done by guessing M^{→} and solving the forward problem to predict the voltages U(\vec{x},t) that would result from this guess. The mapping that takes the total cardiac vector, \vec{M}, to the voltages on the body's surface electrodes, U^{→}, located at x_{I}, I=1, . . . ,L, when U^{→}=[U(x₁,t),U(x₂,t), . . . ,U(x_{L},t)], is denoted by the linear mapping:

$$G ( σ ) {M}^{\text{?}} = {U}^{\text{?}} .$$

$$\text{?} \text{indicates text missing or illegible when filed}$$

Let ^{→}V denote the L component vector of ECG voltages measured on the L electrodes by the ACT 5 system. We solve for the vector M^{→} by minimizing the least squares error functional:

$$E ( {M}^{\text{?}} ) = {‖ {U}^{\text{?}} - {V}^{\text{?}} ‖}_{2} .$$

$$\text{?} \text{indicates text missing or illegible when filed}$$

Total cardiac vector reconstructions were made from the same ACT 5 data set that was used to make the EIT reconstructions shown in FIGS. 11-14.

FIG. 15A is a graph 150 of the comparison of the total cardiac vector paths for a single subject when simultaneously collecting EIT and ECG data. The vectors in each case, m^{→}(tᵢ) where tᵢ₊₁=tᵢ+Δt for Δt=1/864 s are shown during five consecutive cardiac cycles. The path of the total cardiac vector during each cardiac cycle is represented by a unique marker in the plots. The darkest points correspond to atrial depolarization, lighter dots to ventricular depolarization and dark to ventricular repolarization. The lead II electrocardiogram in each case, taken to be the voltage difference between electrodes 16 and 25 are shown below each diagram. The path of the total cardiac vector during a given cardiac cycle is represented by FIG. 15B, which is a graph 152 of the comparison of the total cardiac vector paths for a single subject when collecting only ECG data. FIGS. 15A-15B show the paths traced out by total cardiac vectors \vec{M}(t) reconstructed using both the simultaneously measured ECG and EIT data, as well as the paths reconstructed using only the ECG data. The reconstructions are computed over approximately five cardiac cycles using a cylindrical domain to model the subject's torso. The dipole source's origin was placed at a point within the heart.

The heart's location was determined by correlating the changes in conductivity at each voxel over time with the ECG voltage difference between electrodes 16 and 25 (FIG. 8A). In the case where both the simultaneously measured EIT and ECG data were used, the conductivity at each time t in the reconstruction was assumed to be constant throughout the torso so that σ(\vec{x},t)≈σ₀(t). The value σ₀(t), is defined at each time t to be the constant that best fits the EIT data. In the case where only the ECG data was used, the conductivity was assumed to be fixed at σ₀=0.2 S/m.

In both cases, FIGS. 15A-15B show that the path of the total cardiac vector is largely consistent over multiple cardiac cycles. There are clusters of dark dots 154, 156 corresponding to atrial depolarization, large loops of light dots corresponding to ventricular depolarization, and loops of dark dots corresponding to ventricular repolarization. The path of the total cardiac vector is shown over five cardiac cycles and largely follows the same path for each individual cardiac cycle. When using the best constant conductivity fit to the EIT data σ₀(t) for each frame, the magnitude of the total cardiac vector changes in direct proportion to σ₀(t).

It can be seen that, in one embodiment, the invention provides a method for collecting electrocardiogram (ECG) signals and electrical impedance tomography (EIT) measurements from the same electrodes (82,84) on a patient, by applying, from a computer platform (block diagram 70, FIG. 7), a set of currents to a plurality of electrodes (82,84), the plurality of electrodes in communicative connection to the computer platform (FIG. 7), the plurality of electrodes placed on a patient. Then applying, from the computer platform, a set of currents to the plurality of electrodes to acquire both ECG and EIT signals therefrom, as shown in FIG. 7, and described herein. The method then includes producing, at the computer platform (FIG. 7), one or more diagnostic images of the patient, such as waveforms 106, diagnostic images as shown in FIGS. 11-14, or other graphs and medically relevant data.

As used herein, the term “computing platform” is the hardware infrastructure on which software is executed. For example, in a single computer system, this would be the computer's architecture, operating system (OS), and runtime libraries. In the case of an application program, the most relevant layer is the OS, which can be called a platform itself, such as Unix, Linux, DOS, Windows, or MacOS. In a multi-computer system, such as in the case of offloading processing, it would encompass both the host computer's hardware, operating system (OS), and runtime libraries along with other computers utilized for processing that are accessed via application programming interfaces or a web browser. In hardware, examples of a computer platform are ARM architecture, PC architecture, ASRock, or AMD. In general, as long as it is a required component for a program code to execute, it is part of the computing platform.

The corresponding structures, materials, acts, and equivalents of all means or step plus function elements in the claims below, if any, are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of one or more aspects of the invention and the practical application, and to enable others of ordinary skill in the art to understand one or more aspects of the invention for various embodiments with various modifications as are suited to the particular use contemplated.

What is claimed is:

1. A system for collecting electrocardiogram (ECG) signals and electrical impedance tomography (EIT) measurements from the same electrodes on a patient, comprising:
a computer platform; and
a plurality of electrodes in communicative connection to the computer platform, the plurality of electrodes configured to be placed on a patient,
wherein computer platform further configured to apply a set of currents to the plurality of electrodes to acquire both ECG and EIT signals and produce one or more diagnostic images of the patient.

2. The system of claim 1, wherein the computer platform is further configured to provide a parallel-drive EIT system configured to apply the set of currents to the plurality of electrodes simultaneously.

3. The system of claim 2, wherein the one or more diagnostic images are comprised of a plurality of frames

4. The system of claim 3, wherein the computer platform is further configured to:
apply alternating current to the plurality of electrodes at frequencies in a range from 5 kHz to 500 kHz to create resulting voltages at the plurality of electrodes; and
measure resulting voltages on all electrodes simultaneously at a frame rate of about 27 frames/second.

5. The system of claim 1, wherein the plurality of electrodes are between 16 and 32 electrodes.

6. The system of claim 1, wherein the computer platform further configured to
apply an orthogonal current pattern to each of the plurality of electrodes;
measure a voltage at each electrode for each orthogonal current pattern to create a plurality of measured voltages; and
produce at least one image frame based upon the plurality of measured voltages.

7. The system of claim 3, wherein the computer platform further configured for, for each frame of image, applying a set of orthogonal current patterns wherein each pattern defines the current for each electrode, to produce data for one diagnostic image.

8. The system of claim 1, wherein the computer platform further configured to apply a current waveform to each electrode, each waveform consisting of a series of sinusoidal bursts with each burst having an amplitude and phase corresponding to a current to be applied to that electrode for at least one pattern.

9. The system of claim 1, wherein the computer platform further configured to reduce the noise while producing a flat, high impedance in the bandwidth of the ECG signal.

10. The system of claim 9, wherein the computer platform further including an adaptive filter to remove a tone at a sinusoid frequency to remove the tones at a frame rate and a relative harmonics.

11. A method for collecting electrocardiogram (ECG) signals and electrical impedance tomography (EIT) measurements from the same electrodes on a patient, comprising:
applying, from a computer platform, a set of currents to a plurality of electrodes, the plurality of electrodes in communicative connection to the computer platform, the plurality of electrodes placed on a patient;
applying, from the computer platform, a set of currents to the plurality of electrodes to acquire both ECG and EIT signals therefrom; and
producing, at the computer platform, one or more diagnostic images of the patient.

12. The method of claim 11, further providing, at the computer platform, the set of currents to the plurality of electrodes simultaneously.

13. The method of claim 11, wherein producing the one or more diagnostic images is producing a plurality of frames

14. The method of claim 13, further comprising:
applying, at the computer platform, alternating current to the plurality of electrodes at frequencies in a range from 5 kHz to 500 kHz to create resulting voltages at the plurality of electrodes; and
measuring, at the computer platform, resulting voltages on all electrodes simultaneously at a frame rate of about 27 frames/second.

15. The method of claim 11, further comprising:
applying, at the computer platform, an orthogonal current pattern to each of the plurality of electrodes;
measuring, at the computer platform, a voltage at each electrode for each orthogonal current pattern to create a plurality of measured voltages; and
producing, at the computer platform, at least one image frame based upon the plurality of measured voltages.

16. The method of claim 13, further comprising applying, at the computer platform:
for each frame of image, a set of orthogonal current patterns wherein each pattern defines the current for each electrode; and
producing data for one diagnostic image.

17. The method of claim 11, further comprising applying, at the computer platform, a current waveform to each electrode, each waveform consisting of a series of sinusoidal bursts with each burst having an amplitude and phase corresponding to a current to be applied to that electrode for at least one pattern.

18. A device for collecting electrocardiogram (ECG) signals and electrical impedance tomography (EIT) measurements from the same electrodes on a patient, comprising a computer platform that is in communicative connection to a plurality of electrodes placed on a patient, wherein computer platform further configured to:
simultaneously apply a set of alternating currents to the plurality of electrodes simultaneously to acquire both ECG and EIT signals; and
produce one or more diagnostic images of the patient based upon the acquired ECG and EIT signals.

19. The device of claim 18, wherein the computer platform further including at least one resistor placed in parallel with a source and limiting a maximum impedance.

20. The device of claim 18, wherein the computer platform further including an adaptive filter to remove a tone of known frequency.
